(19) United States
(12) Patent Application Publication
(10) Pub. No.: US 2025/0025698 A1
(43) Pub. Date: Jan. 23, 2025

(54) SYSTEM AND PROCESS FOR CLOSED-LOOP DEEP BRAIN STIMULATION

(71) Applicant: The Cleveland Clinic Foundation, Cleveland, OH (US)

(72) Inventors: Kenneth B. BAKER, Chesterland, OH (US); Brett A. CAMPBELL, Garfield Heights, OH (US); Andre G. MACHADO, Beachwood, OH (US)

(21) Appl. No.: 18/713,215

(22) PCT Filed: Jan. 12, 2023
(86) PCT No.: PCT/US2023/060521

§ 371 Date: May 24, 2024

Related U.S. Application Data
(60) Provisional application No. 63/298,896, filed on Jan. 12, 2022.

(51) Int. Cl. A61N 1/36; G16H 10/60; G16H 20/40; G16H 40/63

(52) U.S. Cl. CPC A61N 1/36139; A61N 1/36067; A61N 1/36096; A61N 1/36103; A61N 1/36167; G16H 10/60; G16H 20/40; G16H 40/63

(57) ABSTRACT

A method includes receiving with a controller, neurophysiology activity data; receiving with the controller, biometric data for the patient; identifying with the controller, one or more weighted components of the neurophysiology activity data; assigning with the controller, based on the biometric data for the patient, a weight to each of the one or more weighted components; determining with the controller, based on a trained algorithm applied to the one or more weighted components, whether to apply the DBS; and instructing application with the controller, based on the determination, the DBS.

### CROSS REFERENCE TO RELATED APPLICATIONS

This application is a National Stage Application of International Patent App. No. PCT/US2023/060521, filed Jan. 12, 2023, which claims the benefit of U.S. Provisional Patent App. No. 63/298,896, filed Jan. 12, 2022, the entire disclosures of both of which are hereby incorporated by reference as if set forth in their entirety herein.

### STATEMENT REGARDING FEDERALLY SPONSORED RESEARCH OR DEVELOPMENT

This invention was made with government support under NS100543 awarded by the National Institutes of Health. The government has certain rights in the invention.

### TECHNICAL FIELD

The disclosure is directed to a system for closed-loop deep brain stimulation. The disclosure is further directed to a process for closed-loop deep brain stimulation. The disclosure is further directed to a system for closed-loop deep brain stimulation for stroke rehabilitation. The disclosure is further directed to a process for closed-loop deep brain stimulation for stroke rehabilitation. In aspects of the disclosure, methods and systems are disclosed that identify when and/or how to apply an application of Deep Brain Stimulation (DBS) to a patient.

### BACKGROUND

Electrical stimulation of the nervous system is an established method for providing functional and therapeutic benefit to patients across a variety of disease contexts. A common stimulation therapy is DBS, where an electrode is placed in specific structures within the brain and a current is delivered to the tissue at the site of implantation. The resulting neural activation provides therapeutic benefit to the patient.

Stimulation settings may be guided first by the disease application, device(s), and their location within the brain. Other parameters such as contact selection, stimulation amplitude, and frequency may further fine tune the delivery of current to achieve therapeutic benefit. Many DBS applications utilize chronic stimulation at fixed parameters. However, chronic stimulation at fixed parameters provides only limited benefits to a patient.

Accordingly, what is needed is a system, device, and/or process to address the limitations of chronic stimulation at fixed parameters.

### SUMMARY OF THE DISCLOSURE

The foregoing needs are met, to a great extent, by the disclosure.

In one general aspect, a method includes receiving with a controller, neurophysiology activity data. The method in addition includes receiving with the controller, biometric data for the patient. The method moreover includes identifying with the controller, one or more weighted components of the neurophysiology activity data; assigning with the controller, based on the biometric data for the patient, a weight to each of the one or more weighted components; determining with the controller, based on a trained algorithm applied to the one or more weighted components, whether, when or how to apply the DBS; and instructing application with the controller, based on the determination, the DBS.

In one general aspect, a system includes a controller configured to receive neurophysiology activity data. The system in addition includes the controller is further configured to receive biometric data for the patient. The system moreover includes the controller is further configured to identify one or more weighted components of the neurophysiology activity data; the controller is further configured to assign based on the biometric data for the patient, a weight to each of the one or more weighted components; the controller is further configured to determine, based on a trained algorithm applied to the one or more weighted components, whether to apply the DBS, when to apply the DBS, and/or how to apply the DBS; and the controller is further configured to instruct application of the DBS in response to the trained algorithm.

There has thus been outlined, rather broadly, certain aspects of the disclosure in order that the detailed description thereof herein may be better understood, and in order that the present contribution to the art may be better appreciated. There are, of course, additional aspects of the disclosure that will be described below and which will form the subject matter of the claims appended hereto.

In this respect, before explaining at least one aspect of the disclosure in detail, it is to be understood that the disclosure is not limited in its application to the details of construction and to the arrangements of the components set forth in the following description or illustrated in the drawings. The disclosure is capable of aspects in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the disclosure. It is important, therefore, that the claims be regarded as including such equivalent constructions insofar as they do not depart from the spirit and scope of the disclosure.

### BRIEF DESCRIPTION OF THE DRAWINGS

FIG. 1 illustrates a system that may be used to configure a DBS system according to aspects of the disclosure.

FIG. 2 illustrates exemplary details of a controller according to aspects of the disclosure.

FIG. 3 is a diagram of a closed-loop system according to aspects of the disclosure.

FIG. 4A and FIG. 4B are process flow diagrams illustrating a method for determining whether, when and/or how to apply a DBS to stimulate a cerebellar pathway connecting to a brainstem, a diencephalon, or a cerebrum of a patient to treat a patient in accordance with an aspect of the present disclosure.

FIG. 5A is a flow diagram for a frequency-based stimulation criteria process according to aspects of the disclosure.

FIG. 5B is a flow diagram for a phase-based stimulation criteria process according to aspects of the disclosure.

FIG. 5C is a flow diagram for a spike-based stimulation criteria process according to aspects of the disclosure.

FIG. 5D is a flow diagram for time locked neural signals as a criterion for a stimulation process according to aspects of the disclosure.

FIG. 6 is an illustrated associative stimulation paradigm according to aspects of the disclosure.

### DETAILED DESCRIPTION

Aspects of the disclosure relate to an adaptive approach that may better optimize stimulation to achieve functional and therapeutic benefit. Incorporating physiological activity to guide stimulation in a closed-loop system may better target pathological activity for elimination or facilitate neurological restoration in applications of DBS.

In this regard, post-stroke rehabilitation is one area that may benefit from the application of DBS. Ischemic stroke remains a leading cause of disability with few options for post-stroke treatment beyond physical rehabilitation. Functional reorganization of the area adjacent to the stroke region (i.e., the perilesional cortex) may be crucial for improving motor outcomes after a stroke has occurred. Functional reorganization of the perilesional cortex may occur in response to electrical stimulation. DBS may be used as a post-stroke rehabilitation therapy alongside physical rehabilitation, and may lead to an increase in neural plasticity of the brain. Specifically, stimulation of the dentothalamacortical circuit via the dentate nucleus may increase neural plasticity, and may in turn aid in the functional reorganization of the perilesional cortex adjacent to the ischemic injury. In response to functional reorganization, a post-stroke patient may experience motor improvement.

Promoting neural plasticity may depend on the timing and nature of the delivery of electrical stimulation. Continuous delivery of the stimulation may not be ideal, and may result in tissue fatigue or other treatment setbacks. Further, continuous delivery of electrical stimulation does not allow for adaptive characteristics resulting from the treatment, does not have appreciation for underlying neural activation, and may stimulate the brain at inopportune or ineffective time points. To achieve maximum reorganization of the perilesional cortex, neural plasticity may need to be promoted at precise times to enhance effectiveness and avoid chronic inundation, fatigue, or habituation of the cerebral activity and/or brain activity to the positive effects of stimulation. Physiological and/or behavioral events may be used as a surrogate for when the brain is most receptive to functional reorganization. These events may be used as a time-lock for stimulation (i.e., closed loop) to maximize improvement. The closed loop system may have the ability to adapt stimulation parameters, based on biological feedback of how the system responds. Examples of the stimulation parameters may include, but are not limited to, an amplitude, a pulse width, a frequency or frequencies, a burst rate, a burst count, a phase orientation, a voltage, a current, an on time period, an off time period, a number of pulses, and/or any combination thereof. Additionally, the closed loop system may target neural activity for elimination and/or enhancement. The system can improve effectiveness with improved timing and avoid poor timing that can be ineffective or detrimental to the rehabilitation process, and may allow for application in other disease modalities and rehabilitation contexts.

By modifying the continuous delivery of the DBS using biofeedback, therapeutic efficacy may be improved. The biofeedback may include electrophysiological feedback, mechanical feedback and/or the like, obtained by one or more means including for example, electroencephalography (EEG), electromyography (EMG), electrocorticography (ECoG), motor function measurements, motor execution measurements, and/or the like. A series of steps may be used to identify key indicators of stimulation efficacy for use in a closed-loop system. The DBS system may incorporate any number of these benchmarks, given that certain criteria may be more relevant for one patient compared to another, therefore there may be flexibility in the weight and relationship between metrics. Analysis of a patient's vigilance levels (i.e., active, alert, asleep), phonation, motor activity (i.e. walking or moving arms), motor planning (i.e. electroencephalographic activity consistent with planning a movement of the upper extremity or initiating walk), mood (measured by autonomic indicators, facial features or other measures) and/or other biometric levels may indicate time points when the patient's brain is most receptive to rehabilitative efforts. Examples include adjusting the timing of stimulation relative to phases of motor planning, adjusting the timing of stimulation relative to phases of motor execution, and/or adjusting the timing of recordings of neural activity.

The DBS system as disclosed herein may also be beneficial in cases of stroke including ischemic stroke, hemorrhagic stroke, and/or the like, traumatic brain injury, epilepsy, schizophrenia, obsessive compulsive disorder, Parkinson's disease, essential tremor, major depressive disorder, and/or other neural disorders.

Methods and systems disclosed herein may incorporate neurophysiological activity as a primary input metric into an algorithm to determine when to apply DBS to a patient. Neurophysiological activity may include, but is not limited to local field potentials (i.e., electrocorticography, electroencephalography, and electromyography), single and multi-unit neuronal activity, heart rate, heart rate variability, and muscle response via, for example, electromyography. These signals may be further processed by a data acquisition system that may isolate relevant components of each signal for integration in determining whether or not to apply electrical stimulation. If criteria for applying the electrical stimulation are met, then the DBS may be applied. Additionally, stimulation may be initiated through user input via an external trigger that communicates wirelessly with an internal pulse generator to manually initiate stimulation. A patient or a clinician may be able to manually turn on stimulation while at home or within a clinical context during active use of a rehabilitation device or activity.

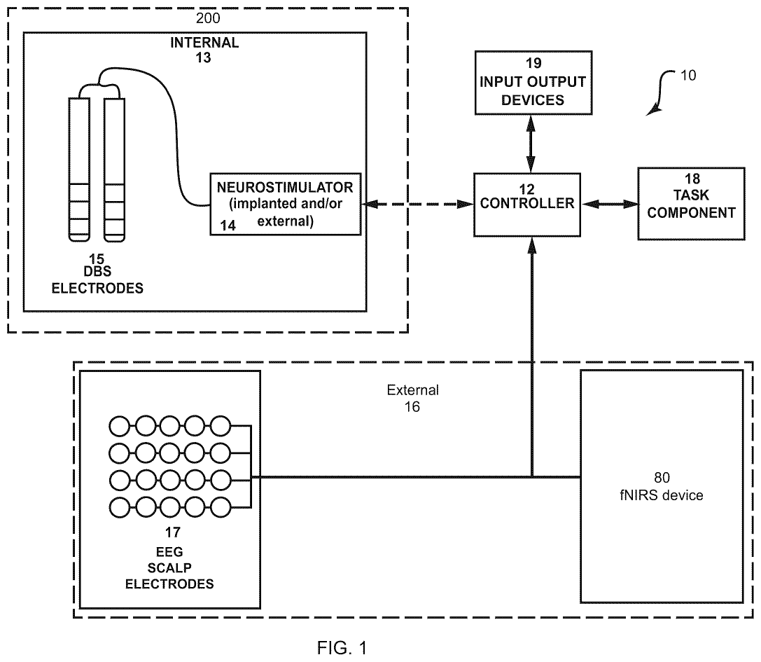

FIG. 1 illustrates a system that may be used to configure a DBS system according to aspects of the disclosure.

In particular, FIG. 1 depicts a system 10 that may be used to configure a DBS system 200 to stimulate a cerebellar pathway connecting to a brainstem, a diencephalon, a cerebrum, and/or the like of a patient to treat a neurological disorder in the patient. The system 10 may be connected to the DBS system 200, the system 10 may be integrated into the DBS system 200, the system 10 may be controlled by the DBS system 200, and/or the like. Further, the system 10 may be implemented as a stimulation determination system, a DBS control system, and/or the like.

The system 10 may include a controller 12. The controller 12 may be configured to receive data from an internal portion 13 of the patient. In certain aspects, the data from the internal portion 13 may be obtained and/or recorded by the one or more DBS electrodes 15. The controller 12 may be configured to receive data from a neurostimulator 14. The neurostimulator 14 may be internal to the body of a patient and/or external to the body of the patient.

Additionally, the controller 12 may be configured to receive data from an external portion 16 of the patient from one or more EEG scalp electrodes 17 (electroencelphalogram electrodes). In certain aspects, the received data may include spontaneous neural activity data received while the patient is at rest. In other aspects, the received data may include data received in response to a patient performing a motor task with a task component 18. The controller 12 may receive data regarding the internal portion 13 via a wired connection, a wireless connection implementing communication channel as defined herein, and/or the like.

Additionally, the controller 12 may be configured to receive data from the external portion 16 with a functional Near-infrared Spectroscopy (fNIRS) device 80. In certain aspects, the fNIRS device 80 measures brain activity by using near-infrared light to estimate cortical hemodynamic activity, which may occur in response to neural activity. In certain aspects, the fNIRS device 80 implements and/or includes a light emitter and a light detector. The light emitter and the light detector of the fNIRS device 80 may be placed on the patient's skull. Further, the light emitter of the fNIRS device 80 may emit light and the light detector may receive the light from the light emitter to generate measurements for the system 10. In certain aspects, data from the external portion 16 also includes invasive recordings, such as data from an implanted electrocorticography (ECoG) strip, an electromyography (not shown), and/or the like that can also communicate with the controller 12.

In aspects, the internal portion 13 may be an implementable system, device, hardware, and/or the like that may implement, at least in part, the controller 12. In aspects, the internal portion 13 may be an implementable ECoG-type system, device, hardware, and/or the like that may implement, at least in part, the controller 12.

The controller 12 may include one or more input output devices 19. In certain aspects, the one or more input output devices 19 may be configured to give instructions to the patient and/or medical professional in response to commands from the system 10 and/or the controller 12. In certain aspects, the one or more input output devices 19 may be configured to provide an output configuration for the DBS system 200. In certain aspects, the one or more input output devices 19 may be configured to control the system 10, the controller 12, the DBS system 200, and/or the like.

In certain aspects, the one or more input output devices 19 may include buttons, soft keys, a mouse, voice actuated controls, a touch screen, a keyboard, a speaker, a microphone, a camera, voice information, and/or the like. The one or more input output devices 19 can be configured to provide outputs from the system 10, the DBS system 200, the controller 12, and/or the like via a graphical user interface, including visual information.

The internal portion 13 may be implanted in a patient, with one or more DBS electrodes 15 in the patient's brain, for example, in contact with or adjacent to the dentate nucleus, and the neurostimulator being remote from the brain (either external to the body or implanted under the patient's skin). In some embodiments, the external portion 16 is not be implanted in the patient. In aspects, the internal portion 13 may include other implantable hardware, such as an ECOG, depth electrodes, other contacts on the DBS lead, and/or the like. Data from the implantable hardware may form part of the feedback as described herein.

The one or more EEG scalp electrodes 17 are illustrated as a plurality of electrodes, but should be understood as including any number of implementations of the one or more EEG scalp electrodes 17 that is limited by the size of the patient's head and greater than one. For example, the number of implementations of the one or more EEG scalp electrodes 17 can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 electrodes, as necessary. The one or more EEG scalp electrodes 17 can be positioned in a 10-5 layout, a 10-10 layout, a 10-20 layout, or the like.

At least one of the components of FIG. 1, such as the controller 12, may be equipped with a non-transitory memory storing instructions for the configuration (and in some instances data) and a processor to access the non-transitory memory and execute the instructions. The non-transitory memory and the processor may be implemented as a single circuit, such as an application specific integrated circuit (ASIC), but may be in any possible implementation of a non-transitory memory and an associated processor. In certain aspects, the one or more input output devices 19 may be implemented as an input device, such as a mouse, a keyboard, and/or the like and may be a component of controller 12 to allow interaction with the controller 12 or any other component the system 10.

The controller 12 may engage in wired and/or wireless communication. For example, the controller 12 may communicate with the neurostimulator 14 that is implanted in an internal portion 13 to the patient's body according to a near field wireless communication means (with any necessary additional circuitry not illustrated). The one or more EEG scalp electrodes 17 may be connected to the controller 12 (through means that may not be illustrated) to engage in wired or wireless communication. The controller 12 can be connected to the task component 18 and/or the one or more input output devices 19 according to a wired or wireless connection.

The task component 18 may be one or more instruments configured to measure one or more mechanical properties of performing a task that the user has been instructed to perform. In certain aspects, the instructions to perform a task may be provided by a physician, generated by the system 10, generated by the controller 12, displayed on the one or more input output devices 19, and/or the like.

As an example, the task component 18 may provide a mechanical or digitized measurement of movement and may include a dynameter, a digital plate, articulated lever, a robotic arm, other mechanical measurement device, a digitized measurement device. This measurement of movement may include, for example, displacement/velocity/acceleration of an extremity or body part, dexterity, strength, resistance (rigidity or spasticity), electromyography, etc., of an extremity or body part. In certain aspects, the task component 18 may measure the movement and provide data regarding the movement to the system 10, the controller 12, and/or the like

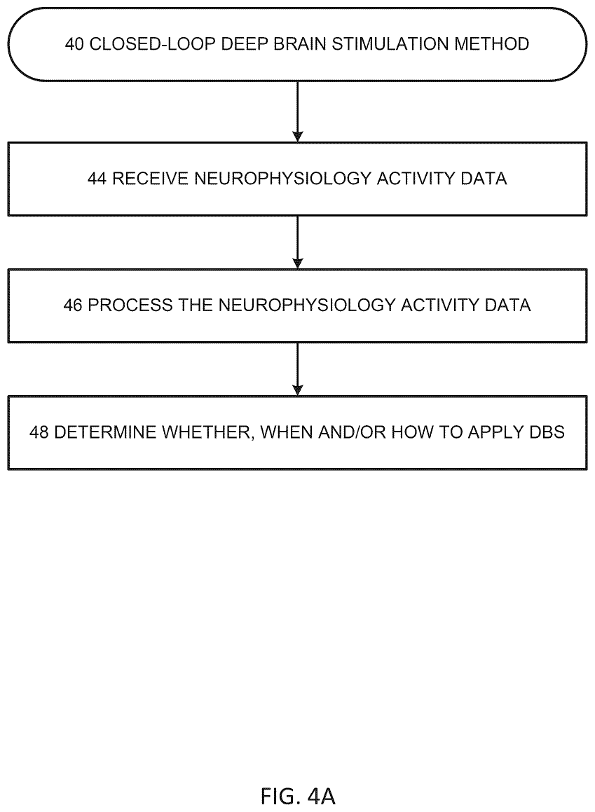

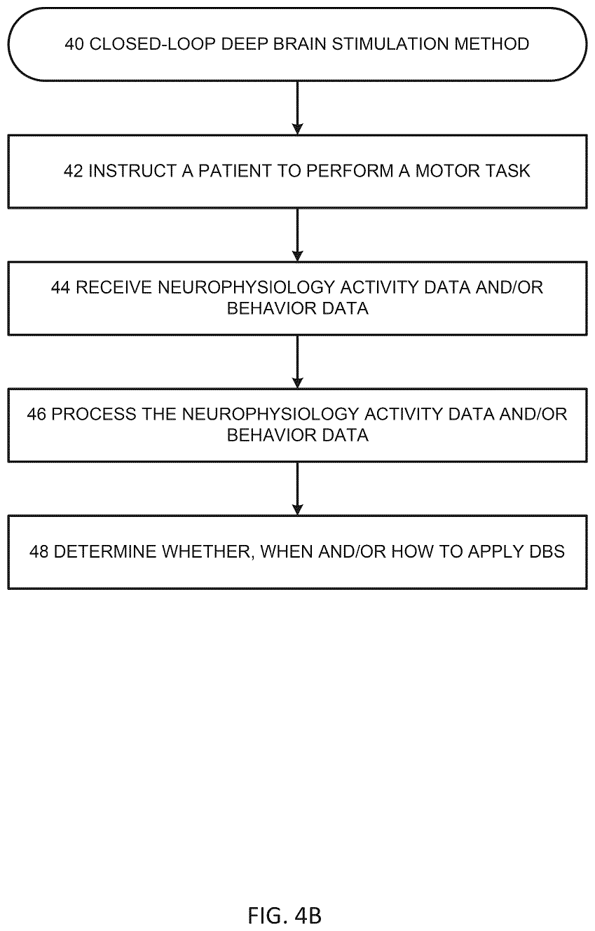

The system 10 may be used to configure a DBS system 200 to stimulate a cerebellar pathway connecting to a brainstem, a diencephalon, a cerebrum, or other location in the brain of a patient to treat a neurological disorder in the patient. The controller 12 may perform steps related to a configuration of the DBS system 200. The configuration of the DBS system 200 may include one or more of: electrical stimulation of any component of a neural pathway associated with the neurological condition; internal recordings of electrophysiology of sub-cortical areas and/or deep brain tissue; external recordings of conduction from the primary motor cortex; secondary motor cortex, primary sensory cortex, and/or secondary sensory cortex; and mechanical measures when performing or attempting to perform at least one task including a motor task, a vocal task, a cognitive task, and/or one or more combinations thereof, with a task component. For example, the system 10 may be used to execute a closed-loop deep brain stimulation method 40 (FIG. 4A and FIG. 4B) described below (or any other process for configuration that uses a different combination of electrical stimulation of any component of a neural pathway associated with the neurological condition, internal recordings of electrophysiology of sub-cortical areas and/or deep brain tissue, external recordings of conduction from the primary motor cortex, secondary motor cortex, primary sensory cortex, and/or secondary sensory cortex, mechanical measures when performing or attempting to perform at least one task, including a motor task, vocal task, cognitive task, and/or the like).

The DBS system 200 may include an implanted pulse generator (IPG). The IPG may be a neurostimulator and may be configured to send electrical pulses to the brain of the patient. The DBS system 200 may include one or more implanted DBS electrodes 15 that may be placed in areas of the brain of the patient. The DBS system 200 may be configured such that the lead may be connected to the IPG.

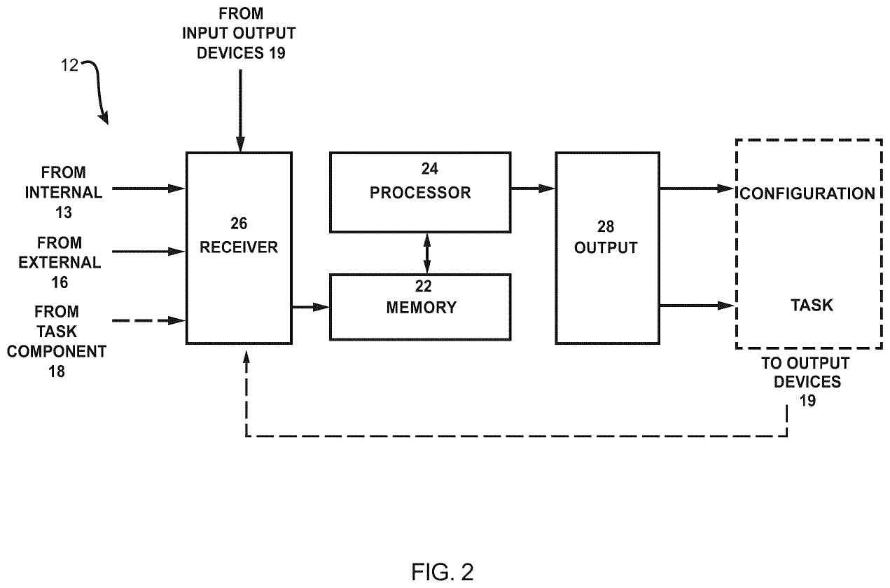

FIG. 2 illustrates exemplary details of a controller according to aspects of the disclosure.

As shown in FIG. 2, the controller 12 may have a non-transitory memory 22 storing instructions, data, and/or the like. For example, the non-transitory memory 22 may store instructions for implementing the closed-loop deep brain stimulation method 40 as further described herein. For example, the non-transitory memory can be a read only memory (ROM), random access memory (RAM), magnetic RAM, core memory, magnetic disk storage medium, optical storage medium, flash memory device, and/or other machine readable mediums (readable by the processor, in other words) for storing information, including instructions and/or data.

The non-transitory memory 22 may be associated with a receiver 26. The receiver 26 may receive data from the internal portion 13, the external portion 16, the task component 18, the one or more input output devices 19 and/or the like. The receiver 26 may receive signals from the internal portion 13 and the external portion 16 that include internal data (e.g., electrophysiology data) and external data (e.g., EEG data). In some instances, the receiver 26 may also receive data from the task component 18, such as information related to one or more mechanical properties of performing a task that the user has been instructed to perform. In some aspects, the one or more input output devices 19 may receive input from the clinician such as limits, constraints, and/or the like for operation of the controller 12.

Additionally, the controller 12 may include a processor 24. The processor 24 may be configured to implement various aspects of the system 10. Moreover, the processor 24 may be configured to implement the closed-loop deep brain stimulation method 40 as further described herein. The processor 24 may be associated with an output 28. The processor 24 may use at least a portion of the data received and provide an output (including a configuration, a task, or the like) to the output 28. The output 28 may provide the output to the one or more input output devices 19, which may provide an audio and/or visual output.

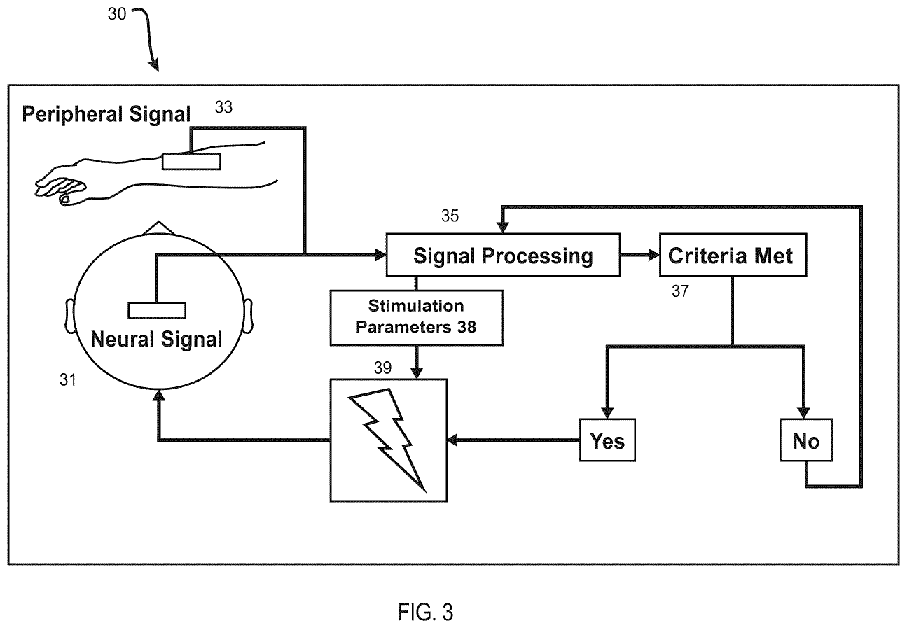

FIG. 3 is a diagram of a closed-loop system according to aspects of the disclosure.

In particular, FIG. 3 is a diagram of a closed-loop system 30. A decision to provide electrical stimulation by the DBS system 200 and/or the manner in which to provide electrical stimulation by the DBS system 200 may be determined based on a closed-loop approach, as opposed to continuous stimulation. In certain aspects, at least one neural signal 31 and/or at least one peripheral signal 33 may be input into a data acquisition platform for signal processing 35. The data acquisition platform may be implemented by the system 10. The at least one neural signal 31 may include, but is not limited to, signals from a local field potentials (LFPs) recorded from an electroencelphalogram (EEG), signals from an electrocorticography (ECOG), and/or signals from another suitable device. The at least one peripheral signal 33 may include, but is not limited to, an output from an electromyography (EMG), a single or multi-unit activity, a heart rate, a heart rate variability, a muscle response, measure of speech performance, such as phonation, gait kinematics, and/or any other physiological aspect of the patient.

The at least one neural signal 31 and the at least one peripheral signal 33 may be processed by the data acquisition platform at a signal processing 35. Here, signal processing techniques are performed to isolate relevant activity contained within the acquired data. Extracted information from the signals may include average response profile, instantaneous power, power with respect to time, distribution of signal frequency, phase components, patient state (i.e., active, alert, asleep, etc.), movement activity levels, and/or the like.

In certain aspects, extracted information may be compared to a set of criteria 37. If the set of criteria 37 to apply the stimulation is met, the system 10 will proceed by applying the DBS 39. If the set of criteria 37 is not met, the closed-loop system 30 will not apply the DBS.

In further aspects, the closed-loop system 30 may have the ability to adapt one or more stimulation parameters for the DBS 39. The stimulation parameters may be based on biological feedback of how the system responds, neurophysiological data to establish the stimulation parameters in relation to on-going neural activity, and/or the like. In other words, the closed-loop system 30 may have the ability to determine how to apply the DBS 39. For example, the system 10, the controller 12, the signal processing 35, and/or the like may generate stimulation parameters 38. Thereafter, the system 10 will proceed by applying the DBS 39 based on the stimulation parameters 38. Here again, examples of the stimulation parameters may include an amplitude of the DBS, a voltage of the DBS, a current of the DBS, a frequency of the DBS, an on time period of the DBS, an off time period of the DBS, a number of pulses of the DBS, a DBS pulse width, a DBS frequency or DBS frequencies, a DBS burst rate, a DBS burst count, a DBS phase orientation, and/or the like.

FIG. 4A and FIG. 4B are process flow diagrams illustrating a method for determining when and/or how to apply a DBS to stimulate a cerebellar pathway connecting to a brainstem, a diencephalon, or a cerebrum of a patient to treat a patient in accordance with an aspect of the present disclosure.

In particular, FIG. 4A and FIG. 4B illustrate a closed-loop deep brain stimulation method 40 that may be executed using the system 10 as shown in FIG. 1 (with further aspects shown in FIG. 2), the controller 12, the DBS system 200, and/or the like. The closed-loop deep brain stimulation method 40 is shown and described as being executed serially; however, it is to be understood and appreciated that the present disclosure is not limited by the illustrated order of the closed-loop deep brain stimulation method 40, as some steps could occur in different orders and/or concurrently with other steps shown and described herein. Moreover, not all illustrated aspects may be required to implement the closed-loop deep brain stimulation method 40, nor is the closed-loop deep brain stimulation method 40 necessarily limited to the illustrated aspects. Additionally, one or more of the steps can be stored in a non-transitory memory and accessed and executed by a processor.

As an optional first step (not shown), an initial monopolar review (or electrical stimulation) can occur to determine any electrode(s) and/or stimulation parameters that cause undesirable side effects. Such information may be input through the one or more input output devices 19 by the clinician to constrain the parameters to those that are not adverse to the particular patient. These electrode(s) and/or stimulation patterns may be excluded from the further steps of the closed-loop deep brain stimulation method 40. The decision to exclude may be specific to the user (e.g., based on symptoms and/or the way the electrodes are implanted). However, the decision to exclude may be based on (or supplemented by) data specific to a population that includes at least one similar patient.

At step 42, which is illustrated in the FIG. 4B closed-loop deep brain stimulation method 40, a patient may be instructed to perform one or more tasks including one or more motor tasks, vocal tasks, cognitive tasks, and the like. For example, the patient may be instructed to perform the one or more tasks (e.g., motor tasks, vocal tasks, cognitive tasks, or the like) by a medical professional. In certain aspects, the one or more motor tasks may be chosen from predefined listing of motor tasks based on the medical condition of the patient. In aspects, step 42 may include obtaining data related to the one or more tasks. In aspects, step 42 may include obtaining data relating to evaluation of how stimulation impacts spontaneous neural data as a distinct option from task-related changes. In aspects, step 42 may include obtaining behavior data. This data may be received from one or more devices and/or from the clinician through the one or more input output devices 19.

As another example, the system 10 and/or the controller 12 may determine the one or motor tasks. In certain aspects, the system 10 and/or the controller 12 may determine the one or tasks (e.g., motor tasks, vocal tasks, cognitive tasks, and/or the like) based on data input into the system 10 and/or the controller 12 about the patient, past performance of the patient, a population of similar patients, and/or the like. Thereafter, the system 10, the controller 12, and/or the like may output the instruction related to the one or more tasks (e.g., motor tasks, vocal tasks, cognitive tasks, and/or the like) by the one or more input output devices 19, an audio output device, a video device and/or the like.

In another example, the system 10 may instruct the patient to perform a task and record the related changes. In response, the patient can perform, at least attempt to perform, plan to perform, think about performing, and/or the like the one or more tasks (e.g., motor tasks, vocal tasks, cognitive tasks, and/or the like) as internal data (e.g., electrophysiology data used in Step 44) and/or external data (e.g., electroencephalography (EEG) data used in Step 46) can be recorded by appropriate electrodes and received (by controller 12). In some instances, the task (e.g., motor task, vocal task, cognitive task, and/or the like) can be aided by the task component 18 of FIG. 1 that can also record data related to the motor task.

Additionally, although described as related to the patient performing or attempting to perform the same task (e.g., motor task, vocal task, cognitive task, and/or the like), it will be understood that the steps can occur with multiple tasks (e.g., motor tasks, vocal tasks, cognitive tasks, and/or the like), which are either the same or different. For example, the motor task can include moving an affected extremity, such as an arm, a hand, a finger, a foot, a leg, and/or the like. In some patients, different parts of the same extremity may be affected and/or different extremities may be affected. Also, the non-affected extremity can be part of the task. Alternatively, the natural movement of the non-affected extremity can be detected by the system 10 and/or the task component 18 for the purposes of detecting motor planning, detecting motor execution, guiding programming and/or stimulation delivery. The vocal task can include repeating a sound or word, reciting a passage, changing tones and/or the like. The cognitive tasks can include one or more memory tasks, calculation tasks and/or other cognitive functions.

In step 44, neurophysiology activity data may be received by the data acquisition platform. The neurophysiology data may include at least one neural signal 31 and/or a peripheral signal 32, and may include signals from local field potentials (LFPs) recorded from an electroencelphalogram (EEG), electrocorticography (ECOG), other suitable device, an output from an electromyography (EMG), and/or the like which may include a single or multi-unit activity, a heart rate, a heart rate variability, a muscle response, and/or the like.

In step 46, the neurophysiology activity data may be processed. Patient-specific data may be isolated from the neurophysiology activity data, using signal processing techniques. Relevant information, such as average response profile, instantaneous power, power with respect to time, distribution of signal frequency, phase components, patient state (i.e., active, alert, asleep, etc.), movement activity levels, etc., may be extracted from the data. The relevant information may be assigned differing weights, based upon the patient or the rehabilitation treatment. The processed signals and their assigned weights may be input into a trained algorithm to determine the outcome of whether to apply the DBS, whether not apply the DBS, when to apply the DBS, and/or how to apply the DBS in step 48.

The algorithm may be implemented with artificial intelligence as defined herein. For example, the algorithm may be a convolutional neural network, a heuristic algorithm, and/or any other suitable algorithm, and may be trained to identify different criteria indicating whether the DBS should be applied, when the DBS should be applied, and/or how the DBS should be applied, such as differing levels of vigilance, or phase of motor planning, for example.

The algorithm may be trained to identify criteria that reflect any other relevant benchmark from the input signals for applying the DBS, such as a frequency, phase, or single-unit or multi-unit activity of the input neural or peripheral signal.

The algorithm may also be trained using a sample dataset from a single patient to differentiate relevant biomarker benchmarks (i.e., whether a patient's eyes are open or closed, whether a patient is awake or asleep, etc.) to determine criteria as to whether to apply the DBS, when to apply the DBS, and/or how to apply the DBS. The algorithm may also be less computationally intensive, allowing for a smaller computing device to be used with the DBS system 200. Alternatively, the algorithm may be trained to determine when to turn off stimulation in cases of continuous input not related to task performance, for example when the patient is at rest.

In certain aspects, the algorithm may utilize any of the input signals to determine how to apply the DBS by the DBS system 200. In other words, the stimulation parameters for application of the DBS by the DBS system 200. For example, the algorithm may determine an amplitude of the DBS, a voltage of the DBS, a current of the DBS, a frequency of the DBS, an on time period of the DBS, an off time period of the DBS, a number of pulses of the DBS, a DBS pulse width, a DBS frequency or DBS frequencies, a DBS burst rate, a DBS burst count, a DBS phase orientation, and/or the like.

In certain aspects, the algorithm may utilize any of the input signals to determine when to apply the DBS by the DBS system 200. For example, the algorithm may determine the timing of when to apply the DBS relative to a specific neural signal, relative to a specific motor task, vocal task, or cognitive task, relative to a pulse wave, relative to a recent DBS application, and the like.

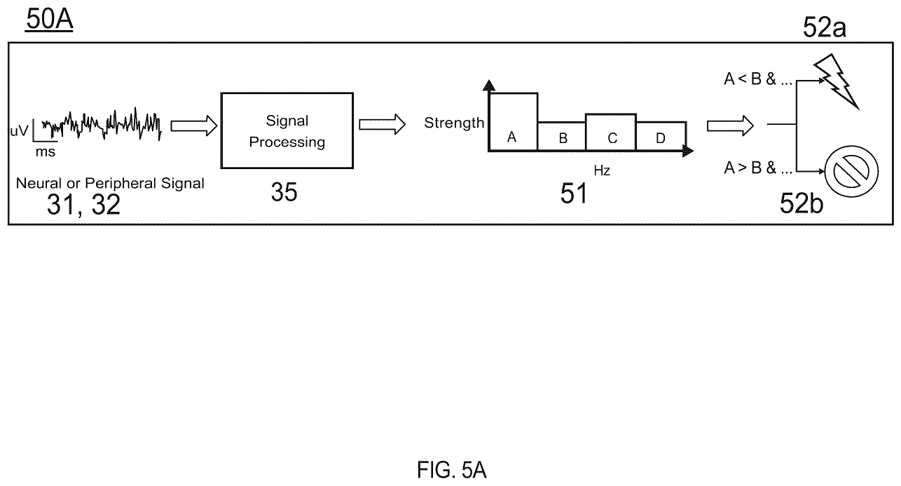

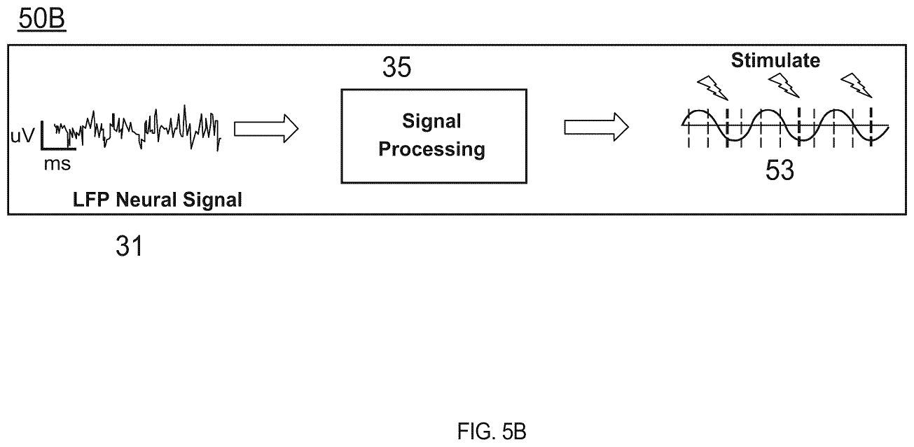

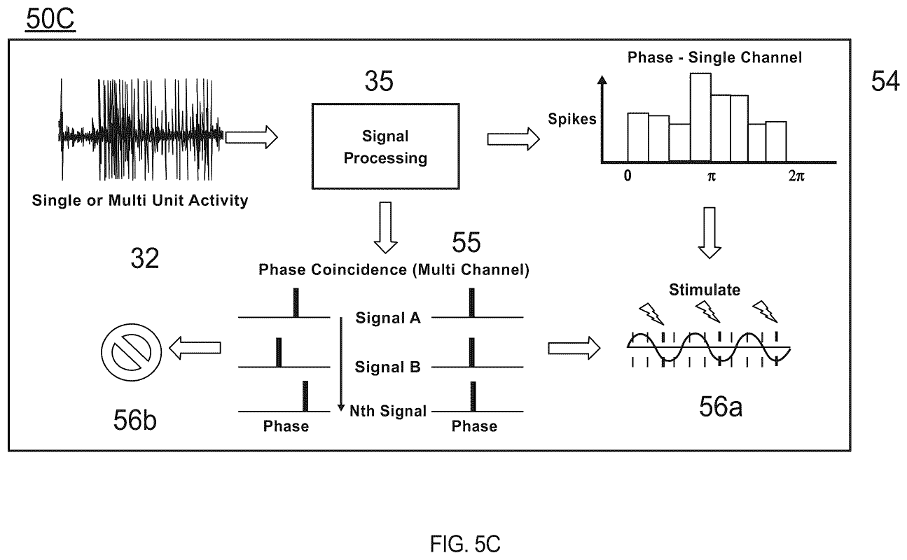

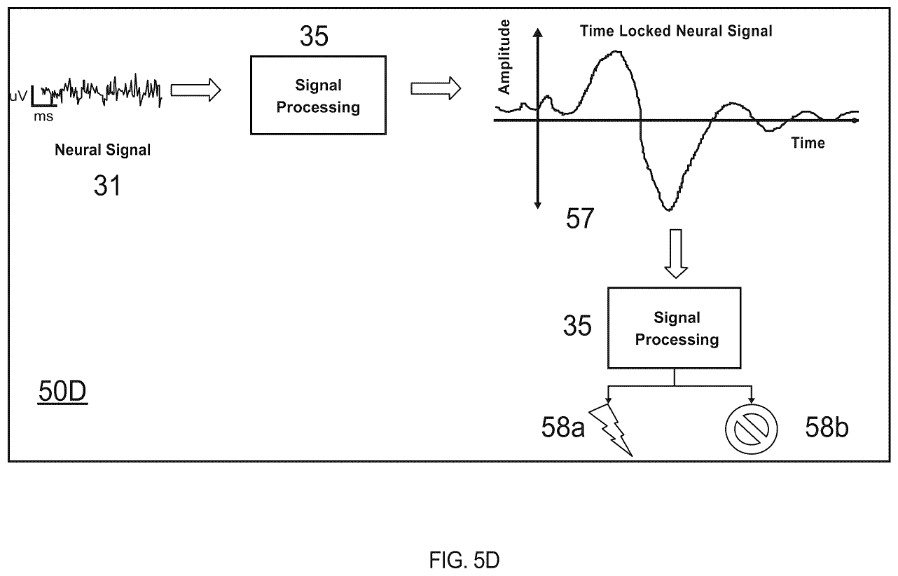

FIGS. 5A-5D are exemplary embodiments of different criteria as benchmarks for application of the DBS. Additional criteria to the benchmarks may also be used in determining whether or not to apply the DBS.

FIG. 5A is a flow diagram for a frequency-based stimulation criteria process according to aspects of the disclosure.

In particular, FIG. 5A is a flow diagram for a frequency-based stimulation criteria process 50A. The data acquisition platform or signal processing 35 may deconstruct one or more frequency component 51 from the at least one neural signal 31 and/or the peripheral signal 32 as criteria for stimulation. For example, if the strength of the first component is less than the subsequent component, this may be within the benchmark, and the DBS is applied at application 52A. If the strength of the first component is greater than the subsequent components, the DBS may not be applied at the no action step 52B. Instantaneous power as well as changes with time in the distribution and/or power of the frequency content are examples of how these data may be used as an input metric.

FIG. 5B is a flow diagram for a phase-based stimulation criteria process according to aspects of the disclosure.

In particular, FIG. 5B is a flow diagram for a phase-based stimulation criteria process 50B. The data acquisition platform or signal processing 35 may process one or more phase components from the at least one neural signal 31 as criteria for stimulation 53. The timing stimulation may be based on the phase of a particular frequency band (or range) to promote or attenuate the resulting response. Avoidance of specific phases in particular frequency bands may also be targeted.

FIG. 5C is a flow diagram for a spike-based stimulation criteria process according to aspects of the disclosure.

In particular, FIG. 5C is a flow diagram for a spike-based stimulation criteria process 50C. The decision to stimulate may incorporate single and/or multi-unit activity from cells recorded in the central or peripheral nervous system, as input by the at least one neural signal 31 to the signal processing 35. A phase-single channel 54 may then determine stimulation 56A. A phase coincidence (multi-channel) 55 may determine either stimulation 56A or no action 56B. The criteria may be based on individual unit activity or the coincidence of multiple cells. Additionally, the phase of the cell firing may be used as a further determining factor for when to stimulate or times to avoid.

FIG. 5D is a flow diagram for time locked neural signals as a criterion for a stimulation process according to aspects of the disclosure.

In particular, FIG. 5D is a flow diagram for time locked neural signals as a criterion for a stimulation process 50D. Activity such as cortical-evoked potentials averaged and time locked to the onset of previous stimulation pulses may serve as an input metric as the at least one neural signal 31 to a signal processing 35. The power, frequency content, and/or other computational metric 57 of the time locked neural signal may be input into the signal processing 35 and into the algorithm's decision to stimulate at stimulation 58A or no action 58B.

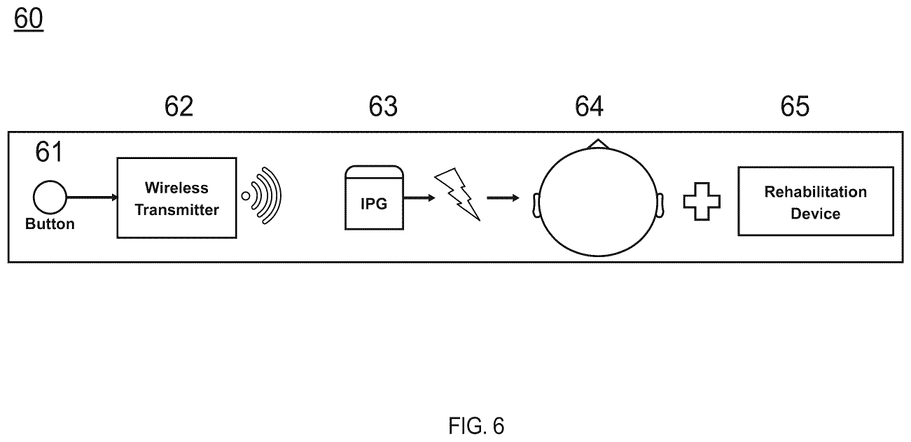

FIG. 6 is an illustrated associative stimulation paradigm according to aspects of the disclosure.

In particular, FIG. 6 is an illustrated associative stimulation paradigm in which a button may activate a wireless transmitter 62, which may be used to trigger stimulation. The wireless transmitter 62 may be paired to an implantable pulse generator (IPG) 63, which applies the stimulation to a patient 64. The IPG may provide chronic (i.e., continuous) stimulation or may engage a specific algorithm to communicate with the rehabilitative device 65 in the absence of or in addition to the physiological inputs. Additionally, the patient 64 may be tasked with the use of a rehabilitation device 65 while completing rehabilitation tasks at home or within a clinical context.

Accordingly, the system 10 and/or the closed-loop deep brain stimulation method 40 may provide an adaptive approach that may better optimize stimulation to achieve functional and therapeutic benefit. In this regard, the system 10 and/or the closed-loop deep brain stimulation method 40 may incorporate physiological activity to guide stimulation in a closed-loop system that may better target pathological activity for elimination or facilitate neurological restoration in applications of DBS.

Additionally, the system 10 and/or the closed-loop deep brain stimulation method 40 may promote neural plasticity by controlling the timing and nature of the delivery of electrical stimulation by the DBS system 200. Continuous delivery of the stimulation may not be ideal, and may result in tissue fatigue or other treatment setbacks. Further, continuous delivery of electrical stimulation does not allow for adaptive characteristics resulting from the treatment, does not have appreciation for underlying neural activation, and may stimulate the brain at inopportune or ineffective time points.

Additionally, the system 10 and/or the closed-loop deep brain stimulation method 40 may achieve maximum reorganization of the perilesional cortex, neural plasticity by promoting at precise times to enhance effectiveness and avoid chronic inundation, fatigue, or habituation of the cortex to the positive effects of stimulation. Additionally, the system 10 and/or the closed-loop deep brain stimulation method 40 may utilize physiological and/or behavioral events as a surrogate for when the brain is most receptive to functional reorganization. These events may be used as a time-lock for stimulation (i.e., closed loop) to maximize improvement.

Additionally, the system 10 and/or the closed-loop deep brain stimulation method 40 may have the ability to adapt stimulation parameters, based on biological feedback of how the system responds. Additionally, the system 10 and/or the closed-loop deep brain stimulation method 40 may target neural activity for elimination and/or enhancement. The system 10 and/or the closed-loop deep brain stimulation method 40 can improve effectiveness with improved timing and avoid poor timing that can be ineffective or detrimental to the rehabilitation process, and may allow for application in other disease modalities and rehabilitation contexts.

Additionally, the system 10 and/or the closed-loop deep brain stimulation method 40 may modify the continuous delivery of the DBS using biofeedback such that therapeutic efficacy may be improved. Additionally, the system 10 and/or the closed-loop deep brain stimulation method 40 may implement series of steps to identify key indicators of stimulation efficacy for use in a closed-loop system. Additionally, the system 10 and/or the closed-loop deep brain stimulation method 40 may incorporate any number of these benchmarks, given that certain criteria may be more relevant for one patient compared to another, therefore there may be flexibility in the weight and relationship between metrics. Additionally, the system 10 and/or the closed-loop deep brain stimulation method 40 may analyze a patient's vigilance levels (i.e., active, alert, asleep), phonation or other biometric levels to determine time points when the patient's brain is most receptive to rehabilitative efforts. Additionally, the system 10 and/or the closed-loop deep brain stimulation method 40 may adjust the timing of stimulation relative to phases of motor planning and/or adjusting the timing of stimulation relative to phases of motor execution.

Additionally, the system 10 and/or the closed-loop deep brain stimulation method 40 may also be beneficial in cases of ischemic stroke, traumatic brain injury, epilepsy, schizophrenia, obsessive compulsive disorder, Parkinson's disease, essential tremor, major depressive disorder, and/or other neural disorders.

Accordingly, the disclosure as set forth implementations of the system 10 and/or the closed-loop deep brain stimulation method 40 to address the limitations of chronic stimulation at fixed parameters.

The following are a number of nonlimiting EXAMPLES of aspects of the disclosure.

One EXAMPLE includes: a method that includes receiving with a controller, neurophysiology activity data. The method in addition includes receiving with the controller, biometric data for the patient. The method moreover includes identifying with the controller, one or more weighted components of the neurophysiology activity data; assigning with the controller, based on the biometric data for the patient, a weight to each of the one or more weighted components; determining with the controller, based on a trained algorithm applied to the one or more weighted components, whether to apply the DBS; and instructing application with the controller, based on the determination, the DBS.

The above-noted EXAMPLE may further include any one or a combination of more than one of the following EXAMPLES: The method of the above-noted EXAMPLE may include: receiving feedback data following the DBS with the controller; training an algorithm based on the feedback data to determine whether a sufficient level of criteria to stimulate the DBS are met with the controller; and instructing application with the controller, based on the determination, the DBS. The method of the above-noted EXAMPLE where the instructing application may include adjusting a timing of a stimulation relative to one or more phases of motor planning and/or relative to one or more phases of motor execution. The method of the above-noted EXAMPLE. The method of the above-noted EXAMPLE where the neurophysiology activity data includes at least one of local field potentials, single and multi-unit neuronal activity, a heart rate, a heart rate variability, and/or a muscle response. The method of the above-noted EXAMPLE where the biometric data for the patient includes at least one of a state of awareness of the patient and/or an activity level of the patient. The method of the above-noted EXAMPLE where determining whether to apply the DBS may include identifying at least one of a benchmark for at least one of a frequency of the received neurophysiological data, a phase of a frequency band of the received neurophysiological data, a spike of individual unit activity, phase coincidence of multi-unit activity of the received neurophysiological data, and/or a time-locked neural signal. The method of the above-noted EXAMPLE may include instructing the patient to complete a task. The method of the above-noted EXAMPLE where the task may include one or more motor tasks. The method of the above-noted EXAMPLE where the one or more motor tasks may include moving an affected extremity. The method of the above-noted EXAMPLE where the trained algorithm may include one or more of a convolutional neural network algorithm and a heuristic algorithm. The method of the above-noted EXAMPLE where the biometric data is collected when the patient has been instructed to complete a task, is in a process of completing a task, and/or has completed a task. The method of the above-noted EXAMPLE where the patient has one or more conditions having ischemic stroke, traumatic brain injury, epilepsy, schizophrenia, obsessive compulsive disorder, Parkinson's disease, essential tremor, major depressive disorder, or other neural disorders.

One EXAMPLE includes: a system that includes a controller configured to receive neurophysiology activity data. The system in addition includes the controller is further configured to receive biometric data for the patient. The system moreover includes the controller is further configured to identify one or more weighted components of the neurophysiology activity data; the controller is further configured to assign based on the biometric data for the patient, a weight to each of the one or more weighted components; the controller is further configured to determine, based on a trained algorithm applied to the one or more weighted components, whether to apply the DBS; and the controller is further configured to instruct application of the DBS in response to the trained algorithm.

The above-noted EXAMPLE may further include any one or a combination of more than one of the following EXAMPLES: The system of the above-noted EXAMPLE may include: the controller is further configured to receive feedback data following the DBS; the controller is further configured to train an algorithm based on the feedback data to determine whether a sufficient level of criteria to stimulate the DBS are met; and the controller is further configured to apply, based on the determination, the DBS. The system of the above-noted EXAMPLE where the controller is further configured to adjust a timing of a stimulation relative to one or more phases of motor planning or relative to one or more phases of motor execution. The system of the above-noted EXAMPLE where the neurophysiology activity data includes at least one of local field potentials, single and multi-unit neuronal activity, a heart rate, a heart rate variability, and/or a muscle response. The system of the above-noted EXAMPLE where the biometric data for the patient includes at least one of a state of awareness of the patient and/or an activity level of the patient. The system of the above-noted EXAMPLE where the controller is further configured to determine whether to apply the DBS may include identifying at least one of a benchmark for at least one of a frequency of a received neurophysiological data, a phase of a frequency band of the received neurophysiological data, a spike of individual unit activity, phase coincidence of multi-unit activity of the received neurophysiological data, and/or a time-locked neural signal. The system of the above-noted EXAMPLE where the controller is further configured to instruct the patient to complete a task. The system of the above-noted EXAMPLE where the task may include one or more motor tasks. The system of the above-noted EXAMPLE where the one or more motor tasks may include moving an affected extremity. The system of the above-noted EXAMPLE where the trained algorithm may include one or more of a convolutional neural network algorithm and a heuristic algorithm. The system of the above-noted EXAMPLE where the controller is further configured such that the biometric data is collected when the patient has been instructed to complete a task, is in a process of completing a task, or has completed a task. The system of the above-noted EXAMPLE where the patient has one or more conditions having ischemic stroke, traumatic brain injury, epilepsy, schizophrenia, obsessive compulsive disorder, Parkinson's disease, essential tremor, major depressive disorder, or other neural disorders.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the disclosure. As used herein, the term “and/or” includes any and all combinations of one or more of the associated listed items.

The terminology used herein is for the purpose of describing particular aspects only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

Aspects of the disclosure may include communication channels that may be any type of wired or wireless electronic communications network, such as, e.g., a wired/wireless local area network (LAN), a wired/wireless personal area network (PAN), a wired/wireless home area network (HAN), a wired/wireless wide area network (WAN), a campus network, a metropolitan network, an enterprise private network, a virtual private network (VPN), an internetwork, a backbone network (BBN), a global area network (GAN), the Internet, an intranet, an extranet, an overlay network, Near field communication (NFC), a cellular telephone network, a Personal Communications Service (PCS), using known protocols such as the Global System for Mobile Communications (GSM), CDMA (Code-Division Multiple Access), GSM/EDGE and UMTS/HSPA network technologies, Long Term Evolution (LTE), 5G (5th generation mobile networks or 5th generation wireless systems), WiMAX, HSPA+, W-CDMA (Wideband Code-Division Multiple Access), CDMA2000 (also known as C2K or IMT Multi-Carrier (IMT-MC)), Wireless Fidelity (Wi-Fi), Bluetooth, and/or the like, and/or a combination of two or more thereof. The NFC standards cover communications protocols and data exchange formats, and are based on existing radio-frequency identification (RFID) standards including ISO/IEC 14443 and FeliCa. The standards include ISO/IEC 18092 [3] and those defined by the NFC Forum

The disclosure may be implemented in any type of computing devices, such as, e.g., a desktop computer, personal computer, a laptop/mobile computer, a personal data assistant (PDA), a mobile phone, a tablet computer, cloud computing device, and the like, with wired/wireless communications capabilities via the communication channels.

It should also be noted that the software implementations of the disclosure as described herein are optionally stored on a tangible storage medium, such as: a magnetic medium such as a disk or tape; a magneto-optical or optical medium such as a disk; or a solid state medium such as a memory card or other package that houses one or more read-only (non-volatile) memories, random access memories, or other re-writable (volatile) memories. A digital file attachment to email or other self-contained information archive or set of archives is considered a distribution medium equivalent to a tangible storage medium. Accordingly, the disclosure is considered to include a tangible storage medium or distribution medium, as listed herein and including art-recognized equivalents and successor media, in which the software implementations herein are stored.

Additionally, the various aspects of the disclosure may be implemented in a non-generic computer implementation. Moreover, the various aspects of the disclosure set forth herein improve the functioning of the system as is apparent from the disclosure hereof. Furthermore, the various aspects of the disclosure involve computer hardware that it specifically programmed to solve the complex problem addressed by the disclosure. Accordingly, the various aspects of the disclosure improve the functioning of the system overall in its specific implementation to perform the process set forth by the disclosure and as defined by the claims.

The artificial intelligence and/or machine learning may utilize any number of approaches including one or more of a convolutional neural network, a heuristic algorithm, cybernetics and brain simulation, symbolic, cognitive simulation, logic-based, anti-logic, knowledge-based, sub-symbolic, embodied intelligence, computational intelligence and soft computing, machine learning and statistics, and/or the like.

Voice recognition software may be utilized in various aspects of the systems and methods. Users may be able to vocalize, rather than utilizing other input processes. For example, the voice recognition software may be configured for generating text from voice input from a microphone or other voice input. A speech signal processor may convert speech signals into digital data that can be processed by the processor. The processor may perform several distinct functions, including serving as the speech event analyzer, the dictation event subsystem, the text event subsystem, and the executor of the application program. The speech signal processor may generate speech event data and transmit this data to the processor to be processed first by the speech event analyzer. The speech event analyzer may generate a list or set of possible candidates among the system recordings that represent or match the voice input processed by the speech signal processor. The speech event analyzer may transmit the candidate sets to a dictation event subsystem. The dictation event subsystem may analyze the candidate sets and choose the best match candidate with the highest degree of similarity. This candidate is then considered the correct translation, and the dictation event subsystem forwards the translation to the text event subsystem which in turn inputs the translated text into the device.

The many features and advantages of the disclosure are apparent from the detailed specification, and, thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

1. A method for identifying when and/or how to apply deep brain stimulation (DBS) to a patient, the method comprising:
receiving with a controller, neurophysiology activity data;
receiving with the controller, biometric data for the patient;
identifying with the controller, one or more weighted components of the neurophysiology activity data;
assigning with the controller, based on the biometric data for the patient, a weight to each of the one or more weighted components;
determining with the controller, based on a trained algorithm applied to the one or more weighted components, whether, when and/or how to apply the DBS; and
instructing application with the controller, based on the determination, the DBS.

2. The method of claim 1, further comprising:
receiving feedback data following the DBS with the controller;
training an algorithm based on the feedback data to determine whether a sufficient level of criteria to stimulate the DBS are met with the controller; and
instructing application with the controller, based on the determination, the DBS.

3. The method of claim 1, wherein the neurophysiology activity data includes at least one of local field potentials, single and multi-unit neuronal activity, a heart rate, a heart rate variability, and/or a muscle response.

4. The method of claim 1, wherein the biometric data for the patient includes at least one of a state of awareness of the patient and/or an activity level of the patient.

5. The method of claim 1, wherein determining whether to apply the DBS comprises identifying at least one of a benchmark for at least one of a frequency of the received neurophysiological data, a phase of a frequency band of the received neurophysiological data, a spike of individual unit activity, phase coincidence of multi-unit activity of the received neurophysiological data, and/or a time-locked neural signal.

6. The method of claim 1, further comprising instructing the patient to complete a task.

7. The method of claim 1, wherein the biometric data is collected when the patient has been instructed to complete a task, is in a process of completing a task, and/or has completed a task.

8. The method of claim 6, wherein the task comprises one or more motor tasks.

9. The method of claim 8, wherein the one or more motor tasks comprises moving an affected extremity.

10. The method of claim 1, wherein the biometric data is collected when the patient is at rest.

11. The method of claim 1, wherein the trained algorithm comprises one or more of a convolutional neural network algorithm and a heuristic algorithm.

12. The method of claim 2, wherein the instructing application comprises adjusting a timing of a stimulation relative to one or more phases of motor planning and/or relative to one or more phases of motor execution.

13. The method of claim 1, wherein the patient has one or more conditions comprising ischemic stroke, hemorrhagic stroke, traumatic brain injury, epilepsy, schizophrenia, obsessive compulsive disorder, Parkinson's disease, essential tremor, major depressive disorder, or other neural disorders.

14. A method for treating stroke in a subject comprising applying closed-loop deep brain stimulation according to the methods of claim 1.

15. A system for identifying when and/or how to apply deep brain stimulation (DBS) to a patient, the system comprising:
a controller configured to receive neurophysiology activity data;
the controller is further configured to receive biometric data for the patient;
the controller is further configured to identify one or more weighted components of the neurophysiology activity data;
the controller is further configured to assign based on the biometric data for the patient, a weight to each of the one or more weighted components;
the controller is further configured to determine, based on a trained algorithm applied to the one or more weighted components, whether to apply the DBS; and
the controller is further configured to instruct application of the DBS in response to the trained algorithm.

16. The system of claim 15, further comprising:
the controller is further configured to receive feedback data following the DBS;
the controller is further configured to train an algorithm based on the feedback data to determine whether a sufficient level of criteria to stimulate the DBS are met; and
the controller is further configured to apply, based on the determination, the DBS.

17. The system of claim 15, wherein the neurophysiology activity data includes at least one of local field potentials, single and multi-unit neuronal activity, a heart rate, a heart rate variability, and/or a muscle response.

18. The system of claim 15, wherein the biometric data for the patient includes at least one of a state of awareness of the patient and/or an activity level of the patient.

19. The system of claim 15, wherein the controller is further configured to determine whether to apply the DBS comprises identifying at least one of a benchmark for at least one of a frequency of a received neurophysiological data, a phase of a frequency band of the received neurophysiological data, a spike of individual unit activity, phase coincidence of multi-unit activity of the received neurophysiological data, and/or a time-locked neural signal.

20. The system of claim 15, wherein the controller is further configured to instruct the patient to complete a task.

21. The system of claim 15, wherein the controller is further configured such that the biometric data is collected when the patient has been instructed to complete a task, is in a process of completing a task, or has completed a task.

22. The system of claim 20, wherein the task comprises one or more motor tasks.

23. The system of claim 22, wherein the one or more motor tasks comprises moving an affected extremity.

24. The system of claim 22, wherein the trained algorithm comprises one or more of a convolutional neural network algorithm and a heuristic algorithm.

25. The system of claim 16, wherein the controller is further configured to adjust a timing of a stimulation relative to one or more phases of motor planning or relative to one or more phases of motor execution.

26. The system of claim 15, wherein the patient has one or more conditions comprising stroke, traumatic brain injury, epilepsy, schizophrenia, obsessive compulsive disorder, Parkinson's disease, essential tremor, major depressive disorder, or other neural disorders.

27. The system of claim 15, wherein the biometric data is collected when the patient is at rest.
